(19) United States
(12) Patent Application Publication
(10) Pub. No.: US 2025/0084187 A1
(43) Pub. Date: Mar. 13, 2025

(54) METHODS OF PREVENTING AND TREATING ABNORMAL CALCIUM SPARKS

(71) Applicant: Kai Yuan Xu, Cockeysville, MD (US)

(72) Inventor: Kai Yuan Xu, Cockeysville, MD (US)

(21) Appl. No.: 18/814,202

(22) Filed: Aug. 23, 2024

Related U.S. Application Data
(60) Provisional application No. 63/523,027, filed on Jun. 23, 2023.

(51) Int. Cl. C07K 16/40; A61K 39/00; A61P 43/00

(52) U.S. Cl. CPC C07K 16/40; A61P 43/00; A61K 2039/505

(57) ABSTRACT

Methods to prevent and treat abnormal calcium sparks use antibody agonists or activators that bind to the (Na⁺+K⁺)-ATPase.

### TECHNICAL FIELD

The invention relates to methods for preventing/inhibiting/treating abnormal calcium (Ca²⁺) spark and its complications using peptide vaccines and antibodies (including both endogenous and exogenous specific antibodies) that bind to alpha (α) subunit or beta (β) subunit or binding to both subunits, of the (Na⁺+K⁺)-ATPase (NKA) and increase NKA activity. An antibody that specifically binds to and can increase NKA activity (activation of NKA) is called an NKA agonist or NKA antibody activator. In addition, abnormal Ca²⁺ spark and its complications can also be prevented and treated by administering of antigenic peptides of the NKA α or β subunits that induce the production of endogenous antibodies, which specifically bind to the α or β subunits of NKA.

### BACKGROUND OF INVENTION

Calcium (Ca⁺) spark is a basic property of many types of excitable and non-excitable cells, including cardiac myocytes, muscle cells, nerve cells, brain cells, skeletal and smooth muscle cells. Ca²⁺ sparks depend on the opening of ryanodine receptors, which are embedded in the cell internal side of the endoplasmic and sarcoplasmic reticulum that stores calcium. Normal Ca²⁺ sparks maintain intracellular calcium homeostasis and cellular functions. Abnormal Ca²⁺ sparks is a major pathway linked to different diseases including, but not limited to, life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, and abnormal Ca²⁺ sparks induced-stroke. Abnormal Ca²⁺ sparks are also detected in aged and dying cells, including cardiac myocytes. Critical unmet needs exist to prevent and treat abnormal Ca²⁺ sparks in patients.

NKA is a transmembrane enzyme responsible for the active reciprocal transport of Na⁺ and K⁺ ions across the plasma membrane of all animal cells. NKA comprises two basic subunits: the α subunit and the β subunit. The larger α subunit is a functional subunit, which catalyzes the hydrolysis of ATP for active transport of Na⁺ and K⁺ ions across the plasma membrane; the smaller β subunit does not participate in the catalytic process of the enzyme but instead acts as a specific chaperone that assists the biogenesis and correct membrane insertion of newly synthesized NKA. The α subunit of NKA has three isoforms, including α1, α2 and α3. The β subunit of NKA also has three isoforms including β1, β2 and β3. NKA plays a vitally important role in cell function. Studies have revealed that significantly reduced NKA activity is tightly associated with abnormal Ca²⁺ sparks conditions, demonstrating that protecting and maintaining NKA functional activity from injury is an essential new target for preventing and treating abnormal Ca²⁺ sparks.

### BRIEF SUMMARY OF INVENTION

Surprisingly, the inventor has discovered that the NKA antibody agonists or activators, which target the alpha subunit, the beta subunit, or both NKA subunits, can prevent and treat abnormal Ca²⁺ sparks.

Invented methods to prevent, inhibit, and treat abnormal Ca²⁺ sparks patients involve, but are not limited to, the passive and active immunotherapeutic methods that can potentially prevent, inhibit, and treat calcium overload induced-abnormal Ca²⁺ sparks in cells, including cardiac myocytes. This method involves the NKA antibody activators and their peptide antigens. The binding of the NKA activator to the NKA enzyme inhibits and stops abnormal calcium sparks, recovers normal calcium homeostasis, prevents the disruption of calcium homeostasis/calcium overload-induced various diseased conditions, and prolongs the patient's lifespan. The invention methods inhibit and stop abnormal calcium sparks and calcium overload, recover normal calcium homeostasis in cells, prevent the abnormal calcium sparks induced various diseases, and prolong cell normal function lifespan. Both NKA activator based active and passive immunizations (also called active and passive immunotherapies) can prevent, inhibit, and treat abnormal Ca²⁺ sparks induced/regulated life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, abnormal Ca²⁺ sparks induced-stroke, and abnormal Ca²⁺ sparks in aged and dying cells, and more.

Examples of NKA antibody activators having α or β subunit binding specificity that can be used in the methods of the present invention include, but are not limited to, SSA78 (also referred as Jianye 2), SSA401 (also referred as KX-2), SSA412 (also referred as KX-1), JY2948, and JY421228 polyclonal, monoclonal, humanized and human version antibodies thereof, and fragments thereof. These antibodies are capable of increasing NKA enzymatic activity (activation of NKA), which are described for treating other different diseases and conditions in Patent Publication No. PCT/US2006/012912 and U.S. Pat. Nos. 9,974,842, 9,956,275, 9,790,270, 9,527,923, 9,409,949, 9,416,159, 9,238,695, 9,279,020, 9,040,046, 8,945,555, 8,496,929, 8,435,519, 8,383,111, 7,754,210, 10,053,505, 10,214,583 and 1,028,736, which are herein as reference information for all purposes.

Examples of NKA peptide antigens also can be used in the methods of the invention. Abnormal Ca²⁺ sparks and its complication associated diseases can be treated by administrating of antigenic peptide antigens (or peptide vaccine) of NKA α or β subunits that induce production of endogenous specific NKA antibody activators against the α or β subunits of NKA and increase NKA activity. Such therapeutic NKA peptides and the administration thereof are also described for treating different diseases and conditions in U.S. Pat. Nos. 9,974,842, 9,956,275, 9,790,270, 9,527,923, 9,409,949, 9,416,159, 9,238,695, 9,279,020, 9,040,046, 8,945,555, 8,496,929, 8,435,519, 8,383,111, 7,754,210, 10,053,505, 10,214,583 and 10,287,361, which are herein incorporated as reference information for all purposes.

In a first aspect, the invention thus provides methods for preventing the formation and progression of abnormal Ca²⁺ sparks, and its complications, comprising contacting cell transmembrane enzyme NKA with an antibody having binding specificity for the α or β subunit of NKA. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7, (iii) NKA activator antibodies in a humanized or human versions thereof, or a fragment or derivative thereof, and (iv) antigens of SEQ ID NOs: 3-7 to generating endogenous NKA activator antibody, in a human or polyclonal versions thereof, or a fragment or derivative thereof. The method may be conducted in vitro or in vivo. The method may also be conducted in blood ex vivo.

In a second aspect, the invention provides methods for treating the formation and progression of abnormal Ca²⁺ sparks, and its complications, comprising contacting cell transmembrane enzyme NKA with an antibody having binding specificity for the α or β subunit of NKA. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7, (iii) NKA activator antibodies in a humanized or human versions thereof, or a fragment or derivative thereof, and (iv) using SEQ ID NOs: 3-7 peptide antigen to generate endogenous NKA activator antibodies, in human or polyclonal versions thereof, or a fragment or derivative thereof. The method may be conducted in vitro or in vivo. The method may also be conducted in blood ex vivo.

In a third aspect, the invention provides methods for inhibiting the formation and progression of abnormal Ca²⁺ sparks in a subject comprising administering an effective amount of an antibody having binding specificity for the α or β subunit of NKA to a subject in need thereof. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7, (iii) NKA activator antibodies in a humanized or human versions thereof, or a fragment or derivative thereof, and (iv) using SEQ ID NOs: 3-7 peptide antigen to generate endogenous NKA activator antibodies, in human or polyclonal versions thereof, or a fragment or derivative thereof. The subject may be one that is characterized has having or at being at greater risk than the general population for abnormal Ca²⁺ sparks and its complications.

In a fourth aspect, the invention provides methods for preventing abnormal Ca²⁺ sparks-induced complications in a subject comprising administering an effective amount of an antibody having binding specificity for the α or β subunit of NKA to a subject in need thereof. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7, (iii) NKA activator antibodies in a humanized or human versions thereof, or a fragment or derivative thereof, and (iv) using SEQ ID NOs: 3-7 peptide antigen to generate endogenous NKA activator antibodies, in human or polyclonal versions thereof, or a fragment or derivative thereof. The subject may one that is at greater risk than the general population for, but not limited to, life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, abnormal Ca²⁺ sparks induced-stroke, and abnormal Ca²⁺ sparks in aged and dying cells, or other disease or condition wherein inhibition of abnormal Ca²⁺ sparks would be desirable or necessary.

In a fifth aspect, the invention provides methods for inhibiting abnormal Ca²⁺ sparks-induced complications in a subject comprising administering an effective amount of an antibody having binding specificity for the α subunit of NKA to a subject in need thereof. Exemplary antibodies that may be used in these methods include, but are not limited to, (i) antibodies having binding specificity for the α subunit of NKA, (ii) antibodies having binding specificity for one or more of the peptides antigen or peptide vaccine represented by SEQ ID NOs: 3-7, (iii) NKA activator antibodies in a humanized or human versions thereof, or a fragment or derivative thereof, and (iv) using SEQ ID NOs: 3-7 peptide antigen to generate endogenous NKA activator antibodies, in human or polyclonal versions thereof, or a fragment or derivative thereof. The subject may be one that is characterized has having or at being at greater risk than the general population for one or more of the following diseases and conditions. Exemplary conditions and diseases include, but are not limited to, life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, abnormal Ca²⁺ sparks induced-stroke, abnormal Ca²⁺ sparks in aged and dying cells, including cardiac myocytes, or other disease or condition wherein inhibition of abnormal Ca²⁺ sparks would be desirable or necessary.

In a sixth aspect, the invention provides methods for treating abnormal Ca²⁺ sparks-induced complications in a subject comprising administering an effective amount of an antibody having binding specificity for the α or β subunit of NKA to a subject in need thereof. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7 (iii) NKA activator antibodies in a humanized or human versions thereof, or a fragment or derivative thereof, and (iv) using SEQ ID NOs: 3-7 peptide antigen to generate endogenous NKA activator antibodies, in human or polyclonal versions thereof, or a fragment or derivative thereof. The subject may be one that is characterized has having or at being at greater risk than the general population for one or more of the following diseases and conditions. Exemplary conditions and diseases include, but are not limited to, life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, abnormal Ca²⁺ sparks induced-stroke, abnormal Ca²⁺ sparks in aged and dying cells, or other disease or condition wherein inhibition of abnormal Ca²⁺ sparks would be desirable or necessary.

In a seventh aspect, the invention provides methods for reversing abnormal Ca²⁺ sparks condition and its complications and maintaining cellular Ca²⁺ homeostasis in a subject comprising administering an effective amount of an antibody having binding specificity for the α or β subunit of NKA to a subject in need thereof. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7, (iii) NKA activator antibodies in a humanized or human versions thereof, or a fragment or derivative thereof, and (iv) using SEQ ID NOs: 3-7 peptide antigen to generate endogenous NKA activator antibodies, in human or polyclonal or monoclonal versions thereof, or a fragment or derivative thereof. The subject may be one that is characterized has having or at being at greater risk than the general population for one or more of the following diseases and conditions. Exemplary conditions and diseases include, but are not limited to, life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, abnormal Ca²⁺ sparks induced-stroke, abnormal Ca²⁺ sparks in aged and dying cells, or other disease or condition wherein inhibition of abnormal Ca²⁺ sparks would be desirable or necessary.

In an eighth aspect, the invention provides methods for protecting cellular Ca²⁺ homeostasis function and prolonging life span for patients with abnormal Ca²⁺ sparks condition and its complications in a subject comprising administering an effective amount of an antibody having binding specificity for the α or β subunit of NKA to a subject in need thereof. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7, (iii) NKA activator antibodies in a humanized or human versions thereof, or a fragment or derivative thereof, and (iv) using SEQ ID NOs: 3-7 peptide antigen to generate endogenous NKA activator antibodies, in human or polyclonal or monoclonal versions thereof, or a fragment or derivative thereof. The subject may be one that is characterized has having or at being at greater risk than the general population for one or more of the following diseases and conditions. Exemplary conditions and diseases include, but are not limited to, life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, abnormal Ca²⁺ sparks induced-stroke, abnormal Ca²⁺ sparks in aged and dying cells, or other disease or condition wherein inhibition of abnormal Ca²⁺ sparks would be desirable or necessary.

In each of these aspects, the antibody may be in a pharmaceutical formulation comprising the antibody and a pharmaceutically acceptable carrier.

The foregoing has outlined rather broadly the features and technical advantages of the present invention in order that the detailed description of the invention that follows may be better understood. Additional features and advantages of the invention will be described herein, which form the subject matter of the claims of the invention.

### BRIEF DESCRIPTION OF DRAWINGS

FIG. 1 (FIG. 1). Time course of the therapeutic effect of NKA antibody activator SSA412 on abnormal Ca²⁺ sparks in isolated rat cardiac myocytes. FIG. 1 contains 12 images (FIG. 1A-FIG. 1L) under the absence and presence of NKA antibody activator SSA412 experimental conditions. Confocal imaging was performed using a Zeiss LSM510 confocal microscope (Carl Zeiss Inc., Germany). Confocal image FIG. 1A detected massive abnormal Ca²⁺ sparks generation in Ca²⁺ indicator-treated cardiac myocytes, which cells were left in room temperature for 4 hours. This FIG. 1A confocal image was taken in the absence of NKA antibody activators as a control background for comparison.

FIG. 1B. NKA activator SSA412 (0.5 μM) was added to the same Ca²⁺ indicator-treated cardiac myocytes as shown in FIG. 1A that were left in room temperature for 4 hours. This confocal image was the first image taken in the presence of NKA antibody activator.

FIGS. 1C, 1D, 1E, 1F, 1G, 1H, 1I, 1J, 1K, and 1L. Inhibition time course of abnormal Ca²⁺ sparks by the NKA antibody activator SSA412 in Ca²⁺ indicator-treated cardiac myocytes. Confocal time course images reveal that abnormal Ca²⁺ sparks were gradually inhibited by NKA antibody activator SSA412 and disappeared as shown in FIGS. 1C, 1D, 1E, 1F, 1G, 1H, 1I, 1J, 1K, & 1L. These results provide direct evidence demonstrates that NKA antibody activator SSA412 protected cardiac myocytes cell function by significantly inhibited massive abnormal Ca²⁺ sparks condition.

### DETAILED DESCRIPTION OF THE INVENTION

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As outlined in a general manner above, the present invention is based on the surprising discovery that NKA antibody agonists or activators with α or β subunit activation site binding specificity can be used to prevent, inhibit, and treat abnormal Ca²⁺ sparks and its dangerous complications, and abnormal Ca²⁺ sparks associated diseases and conditions in a subject. Such NKA activator antibodies thus form the basis of methods of inhibiting or treating or preventing abnormal Ca²⁺ sparks associated with diseases, whether in vitro or in vivo, to inhibit, treat, and prevent abnormal Ca²⁺ sparks and its complications in a subject. The NKA antibody activators and their peptide antigens also form the basis of methods of inhibiting, treating, or preventing abnormal Ca²⁺ sparks, and their associated diseases and complications.

### Antibodies

The skilled artisan will understand that the particular attributes of the antibodies that may be used in the methods of the present invention are only confined by (i) the ability to bind with specificity to the α or β subunit of NKA, and (ii) the ability to inhibit abnormal Ca²⁺ sparks and their complications and associated diseases.

As described in PCT/US2006/012912 and U.S. Pat. Nos. 9,974,842, 9,956,275, 9,790,270, 9,527,923, 9,409,949, 9,416,159, 9,238,695, 9,279,020, 9,040,046, 8,945,555, 8,496,929, 8,435,519, 8,383,111, 7,754,210, 10053505, 10214583 and 10287361, five antibodies have been prepared that specifically bind the α or β subunit of NKA for treating different diseases. The following antibodies specifically bind to the α subunit of NKA, namely antibody SSA78 (also referred as Jianye 2 antibody), SSA401 (also referred as KX-2 antibody), and SSA412 (also referred as KX-1 antibody). Antibodies JY2948 and JY421228 specifically bind to the β subunit of NKA. As shown in the Examples below, these antibodies may be used in the methods of the present invention. SSA78 binds to amino acids RSATEEEPPNDD (SEQ ID NO:3), SSA401 binds to amino acids HLLGIRETWDDRWIN (SEQ ID NO:4), SSA412 binds to amino acids DVEDSYGQQWTYEQR (SEQ ID NO:5), JY2948 binds to amino acids KERGEFNHERGER (SEQ ID NO:6), and JY421228 binds to amino acids RDEDKDKVGNIEY (SEQ ID NO:7). The invention therefore provides the use of NKA activator antibodies SSA78, SSA401, SSA412, JY2948, and antibody JY421228 in the methods disclosed herein.

The invention also provides the use of antibodies that specifically bind an epitope of the α or β subunit of NKA comprising the amino acid sequence RSATEEEPPNDD (SEQ ID NO: 3), HLLGIRETWDDRWIN (SEQ ID NO:4), DVEDSYGQQWTYEQR (SEQ ID NO:5), KERGEFNHERGER (SEQ ID NO:6), and RDEDKDKVGNIEY (SEQ ID NO:7), or any combination thereof.

The invention further provides for the use of antibodies having binding specificity for an epitope of the α or β subunit of NKA comprising the amino acid SEQ ID NOs: 3-7. Antibody SSA78 binds to amino acids RSATEEEPPNDD (SEQ ID NO:3), SSA401 binds to amino acids HLLGIRETWDDRWIN (SEQ ID NO:4), SSA412 binds to amino acids DVEDSYGQQWTYEQR (SEQ ID NO:5), JY2948 binds to amino acids KERGEFNHERGER (SEQ ID NO:6), and JY421228 binds to amino acids RDEDKDKVGNIEY (SEQ ID NO:7).

The invention further provides for the use of antibodies having binding specificity for variants of each of the peptides of SEQ ID NOs: 3-7, the variants having 8 or fewer, 5 or fewer, 4 or fewer, 3 or fewer, 2 or fewer, or 1 amino acid change in comparison to the peptides of SEQ ID NOs: 3-7. The changes are each individually selected from insertions, deletions, and substitutions. The substitutions may be conservative or non-conservative amino acid substitutions. Each of the variant peptides maintains the ability to induce production of antibodies that specifically bind the α or β subunit of NKA and that have the ability to inhibit abnormal Ca²⁺ sparks and its complications associated diseases and conditions.

In addition, the invention provides for the use of antibodies having binding specificity for other epitopes of the α and β subunit of NKA, with those antibodies having binding specificity for other epitopes of the α or β subunit of NKA being of particular note.

The antibodies used in the methods of the present invention and defined above may be polyclonal, monoclonal, humanized, chimeric antibodies, or human version, and the antibodies may be in the form of an antiserum comprising the antibodies. The antibodies may be of any class, such as IgG1, IgG2, IgG3, IgG4, IgM, IgA1, IgA2, IgD or IgE. The antibodies may be isolated antibodies, purified antibodies, exogenous antibodies, endogenous antibodies, single chain antibodies, single-chain variable fragment, or a combination thereof.

The antibodies may also be antibody fragments of less than the entire antibody, including, but not limited to, single chain antibodies, F(ab′)₂ fragments, Fab fragments, and fragments produced by an Fab expression library, and derivatives of the antibodies and fragments defined herein, with the only limitation being that the antibody fragments and derivatives retain the ability to bind the α or β subunit and inhibit abnormal Ca²⁺ sparks and its complications associated diseases and conditions. It will thus be clear to the skilled artisan that all references to “antibodies” herein include both full-size antibodies as well as antibody fragments, as defined herein.

The antibodies may be produced in any species of animal, though preferably from a mammal such as a human, simian, mouse, rat, rabbit, guinea pig, horse, cow, sheep, goat, pig, dog or cat. For example, the antibodies can be human antibodies or humanized antibodies, or any antibody preparation suitable for administration to a human. For the production of the antibodies, the selected species of animal can be immunized by injection with one or more of the peptides or variants discussed herein. The peptides and variants may be administered in conjunction with one or more pharmaceutically acceptable adjuvants to increase the immunological response. Suitable adjuvants include, but are not limited to, Freund's Complete and Incomplete Adjuvant, Titermax, Oil in Water Adjuvants, as well as Aluminum compounds where antigens, normally peptides, are physically precipitated with hydrated insoluble salts of aluminum hydroxide or aluminum phosphate. Other adjuvants include liposome-type adjuvants comprising spheres having phospholipid bilayers that form an aqueous compartment containing the peptide and protect it from rapid degradation, and that provide a depot effect for sustained release. Surface active agents may also be used as adjuvants and include lipoteichoic acid of gram-positive organisms, lipid A, and TDM. Quil A and QS-21 (saponin-type adjuvants), monophosphoryl lipid A, and lipophilic MDP derivatives are suitable adjuvants that have hydrophilic and hydrophobic domains from which their surface-active properties arise. Compounds normally found in the body such as vitamin A and E, and lysolecithin may also be used as surface-active agents. Other classes of adjuvants include glycan analog, coenzyme Q, amphotericin B, dimethyldioctadecylammonium bromide (DDA), levamisole, and benzimidazole compounds. The immunostimulation provided by a surface active agent may also be accomplished by either developing a fusion protein with non-active portions of the cholera toxin, exotoxin A, or the heat labile toxin from E. coli. Immunomodulation through the use of anti-IL-17, anti IFN-γ, anti-IL-12, IL-2, IL-10, or IL-4 may also be used to promote a strong Th2 or antibody mediated response to the immunogenic formulation.

Means for preparing antibodies are very well known in the art. The antibodies of the invention can be prepared using any known technique that provides for the production of antibody molecules. Suitable techniques include, but are not limited to, the hybridoma technique originally described by Koehler and Milstein (Nature 256:495-497 (1975)), the human B-cell hybridoma technique (Kosbor et al., Immunol Today 4:72 (1983); Cote et al., Proc Natl. Acad. Sci 80:2026-2030 (1983)), and the EBV-hybridoma technique (Cole et al., Monoclonal Antibodies and Cancer Therapy, Alan R. Liss Inc, New York N.Y., pp 77-96 (1985)). Each of these publications is herein incorporated by reference in its entirety. Additionally, antibodies can be produced by inducing in vivo production in the lymphocyte population or by screening recombinant immunoglobulin libraries or panels of highly specific binding reagents as disclosed in Orlandi et al., Proc Natl. Acad. Sci. USA 86:3833-3837 (1989), and in Winter G. and Milstein C., Nature 349:293-299 (1991), both of which is herein incorporated by reference in its entirety.

Humanized antibodies are those where a human antibody has been engineered to contain non-human complementarity-determining regions (CDRs) derived from an antibody produced in a non-human host against a selected antigen. Means for producing humanized antibodies are well-known in the art and include Vaswani S K, and Hamilton R G, Ann Allergy Asthma Immunol. 81 (2): 105-15 (1998) and Kashmiri S V et al., Methods 36 (1): 25-34 (2005), each of which is herein incorporated by reference in its entirety.

Human antibodies are those where an antibody produced in a non-human host against a selected antigen. Means for producing human antibodies are well-known in the art and include Bruggemann M et al., Arch Immunol Ther Exp (Warsz). 63 (2): 101-108 (2014), Pruzina S et al., Protein Eng Des Sel. 24 (10): 791-799 (2011), and Smith K et al., Nature. 4:372-384 (2009), each of which is herein incorporated by reference in its entirety.

Chimeric antibodies are those where an antigen binding region (e.g., F(ab′)₂ or hypervariable region) of a non-human antibody is transferred into the framework of a human antibody by recombinant DNA techniques. Techniques developed for the production of such antibodies include the splicing of mouse antibody genes to human antibody genes to obtain a molecule with appropriate antigen specificity and biological activity. Such techniques are also well known and include: Morrison et al., Proc Natl. Acad. Sci 81:6851-6855 (1984); Neuberger et al., Nature 312:604-608 (1984); Takeda et al., Nature 314:452-454 (1985), each of which is herein incorporated by reference in its entirety.

Techniques for the production of single chain antibodies are described in in U.S. Pat. No. 4,946,778, incorporated herein by reference in its entirety.

Antibody fragments such as F(ab′)₂ fragments can be produced by pepsin digestion of the antibody molecule, and Fab fragments can be generated by reducing the disulfide bridges of the F(ab′)₂ fragments. Alternatively, Fab expression libraries can be constructed to allow rapid and easy identification of monoclonal Fab fragments with the desired specificity (Huse W. D. et al., Science 256:1275-1281 (1989), herein incorporated by reference in its entirety).

The invention provides for the use of pharmaceutical formulations comprising one or more of the antibodies of the invention and a pharmaceutically acceptable carrier. Such formulations may be administered to a subject when practicing the methods of the present invention. Suitable examples of carriers are well known to those skilled in the art and include water, water-for-injection, saline, buffered saline, dextrose, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes. The terms specifically exclude cell culture medium. The formulations may further comprise stabilizing agents, buffers, antioxidants and preservatives, tonicity agents, bulking agents, emulsifiers, suspending or viscosity agents, inert diluents, fillers, and combinations thereof.

The identity of the carrier(s) will also depend on the means used to administer pharmaceutical formulations comprising antibodies to a subject. For example, pharmaceutical formulations for intramuscular preparations can be prepared where the carrier is water-for-injection, 0.9% saline, or 5% glucose solution. Pharmaceutical formulations may also be prepared as liquid or powdered atomized dispersions for delivery by inhalation. Such dispersion typically contains carriers common for atomized or aerosolized dispersions, such as buffered saline and/or other compounds well known to those of skill in the art. The delivery of the pharmaceutical formulations via inhalation has the effect of rapidly dispersing the vaccine formulation to a large area of mucosal tissues as well as quick absorption by the blood for circulation. One example of a method of preparing an atomized dispersion is described in U.S. U.S. Pat. No. 6,187,344, entitled, “Powdered Pharmaceutical Formulations Having Improved Dispersibility,” which is hereby incorporated by reference in its entirety.

Additionally, the pharmaceutical formulations may also be administered in a liquid form. The liquid can be for oral dosage, for ophthalmic or nasal dosage as drops, or for use as an enema or douche. When the pharmaceutical formulation is formulated as a liquid, the liquid can be either a solution or a suspension of the pharmaceutical formulation. There is a variety of suitable formulations for the solution or suspension of the pharmaceutical formulations that are well known to those of skill in the art, depending on the intended use thereof. Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents.

The invention also includes inhibiting abnormal Ca²⁺ sparks and its complications associated diseases and conditions by administering a peptide vaccine that includes one or more peptides selected from a group of peptides having the amino acid sequences RSATEEEPPNDD (SEQ ID NO:3), HLLGIRETWDDRWIN (SEQ ID NO:4), DVEDSYGQQWTYEQR (SEQ ID NO: 5), KERGEFNHERGER (SEQ ID NO:6), and RDEDKDKVGNIEY (SEQ ID NO:7), and variants or fragments thereof. These peptides represent antigenic determinants or epitopes on the α or β subunit of NKA. The peptide vaccines stimulate the host immune system to generate antibodies against the respective one or more peptide epitopes. Methods of using peptide antigen for making, isolating and purifying NKA activity-increasing antibodies and the above-identified peptide antigenic determinants are described in U.S. Patent Applications 20040057956 and 20030228315, the entire contents of which are hereby incorporated by reference as if fully set forth herein.

### Methods for Inhibiting Abnormal Ca²⁺ Sparks

As indicated above, the present invention includes methods for inhibiting abnormal Ca²⁺ sparks and its complications. This method comprising contacting many types of excitable and non-excitable cells, including cardiac myocytes, muscle cells, nerve cells, brain cells, skeletal and smooth muscle cells with an antibody having binding specificity for the α or β subunit of NKA. It will be apparent to the skilled artisan that this method can be practice in vitro, in vivo and ex vivo.

Any of the antibodies described herein, whether polyclonal or monoclonal, can be used in the method, as well as humanized or chimeric versions or human versions of the antibodies, and fragments of any of these. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7, (iii) antibodies in polyclonal, monoclonal, humanized and human versions thereof, and (iv) antibodies in an exogenous or endogenous versions thereof.

### Methods for Inhibiting Abnormal Ca²⁺ Sparks Induced Complications

The present invention includes methods for inhibiting abnormal Ca²⁺ sparks induced complications. This method comprising contacting cardiac myocytes, or muscle cells, or nerve cells, and or cerebral artery smooth muscle cells with an antibody having binding specificity for the α or β subunit of NKA. It will be apparent to the skilled artisan that this method can be practice in vitro, in vivo and ex vivo.

Any of the antibodies described herein, whether polyclonal or monoclonal, can be used in the method, as well as humanized or human version of the antibodies, and fragments of any of these. Exemplary antibodies that may be used in these methods include, but are not limited to, (i) antibodies having binding specificity for the α or β subunit of NKA, (ii) antibodies having binding specificity for one or more of the peptides represented by SEQ ID NOs: 3-7, (iii) antibodies polyclonal, monoclonal, humanized and human versions thereof, and (iv) antibodies in an exogenous or endogenous versions thereof.

### Methods of Treatment

The invention also provides methods for treating or preventing particular diseases, disorders and conditions in a subject by inhibiting abnormal Ca²⁺ sparks.

The invention thus includes methods for inhibiting abnormal Ca²⁺ sparks complications in a subject comprising administering an effective amount of an antibody having binding specificity for the α or β subunit of NKA to a subject in need thereof. While the subject is not limited to one having a particular disease or condition, the subject may be one that is characterized has having or at being at greater risk than the general population for one or more of the following diseases and conditions: life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, abnormal Ca²⁺ sparks induced-stroke, abnormal Ca²⁺ sparks in aged and dying cells, including cardiac myocytes, or other disease or condition wherein inhibition of abnormal Ca²⁺ sparks would be desirable or necessary.

The invention includes methods for inhibiting, treating, or preventing abnormal Ca²⁺ sparks and its complications in a subject, where the method comprises administering an effective amount of an antibody having binding specificity for the α or β subunit of NKA to a subject in need thereof. While the subject is not limited to one having a particular disease or condition, the subject may be one that is characterized has having or at being at greater risk than the general population for one or more of the following diseases and conditions: life-threatening ventricular arrhythmia, ventricular tachycardia, ventricular fibrillation, prevalent atrial arrhythmias (atrial fibrillation and flutter), heart failure, myocardial infarction, SR Ca²⁺ leak, muscular dystrophy, Ca²⁺ overload, diabetes associated Ca²⁺ signaling dysfunction, hypertension, dysfunction of Ca²⁺ homeostasis in myocardial ischemia/reperfusion injury, abnormal Ca²⁺ sparks induced-stroke, abnormal Ca²⁺ sparks in aged and dying cells, including cardiac myocytes, or other disease or condition wherein inhibition of abnormal Ca²⁺ sparks would be desirable or necessary.

Any of the antibodies described herein, whether polyclonal or monoclonal, can be used in the method, as well as humanized or chimeric or human versions of the antibodies, and fragments and derivatives of any of these. Exemplary antibodies that may be used in these methods include, but are not limited to, (i) antibodies having binding specificity for the α or β subunit of NKA, including isoform of a and β subunit, (ii) antibodies having binding specificity for one or more of the peptides represented by SEQ ID NOs: 3-7, (iii) antibodies in a polyclonal, monoclonal, humanized or human versions thereof, or a fragment, or single-chain antibody, or single-chain variable fragment, or derivative thereof, and (iv) antibodies in an exogenous or endogenous versions thereof, or a fragment or derivative thereof. The antibody may be administered as a pharmaceutical formulation comprising the antibody and a pharmaceutically acceptable carrier.

As used herein, the terms “treat”, “treating” and “treatment” have their ordinary and customary meanings, and include one or more of, ameliorating abnormal Ca²⁺ sparks, ameliorating a symptom of abnormal Ca²⁺ sparks complications, or decreasing in severity and/or frequency a symptom of abnormal Ca²⁺ sparks complications. Treatment means ameliorating or decreasing by about 1% to about 100% versus a subject to which the antibody has not been administered. Preferably, the ameliorating or decreasing or inhibiting is about 100%, about 99%, about 98%, about 97%, about 96%, about 95%, about 90%, about 80%, about 70%, about 60%, about 50%, about 40%, about 30%, about 20%, about 10%, about 5% or about 1%. The treatment may begin prior to, concurrent with, or after the onset of clinical symptoms of abnormal Ca²⁺ sparks and abnormal Ca²⁺ sparks complications. The results of the treatment may be permanent or may continue for a period of days (such as 1, 2, 3, 4, 5, 6 or 7 days), weeks (such as 1, 2, 3 or 4 weeks) or months (such as 1, 2, 3, 4, 5, 6 or more months).

As used herein, the terms “prevent”, “preventing” and “prevention” have their ordinary and customary meanings, and include one or more of, stopping, averting, avoiding or blocking abnormal Ca²⁺ sparks and its complications, the occurrence of a symptom of abnormal Ca²⁺ sparks and its complications, the recurrence of a symptom of abnormal Ca²⁺ sparks and its complications, the development of abnormal Ca²⁺ sparks and its complications or the progression of abnormal Ca²⁺ sparks and its complications. Prevention means stopping by at least about 95% versus a subject to which the antibody has not been administered. Preferably, the stopping is about 100%, about 99%, about 98%, about 97%, about 96% or about 95%. The results of the prevention may be permanent or may continue for a period of days (such as 1, 2, 3, 4, 5, 6 or 7 days), weeks (such as 1, 2, 3 or 4 weeks) or months (such as 1, 2, 3, 4, 5, 6 or more months).

As used herein, the terms “inhibit”, “inhibiting” and “inhibition” have their ordinary and customary meanings, and include one or more of, hindering, impeding, obstructing, deterring or restraining abnormal Ca²⁺ sparks and its complications, the occurrence of a symptom of abnormal Ca²⁺ sparks and its complications, the recurrence of a symptom of abnormal Ca²⁺ sparks and its complications, the development of abnormal Ca²⁺ sparks and its complications, or the progression of abnormal Ca²⁺ sparks and its complications. Inhibition means impeding by about 1% to about 100% versus a subject to which the antibody has not been administered. Preferably, the impeding is about 100%, about 99%, about 98%, about 97%, about 96%, about 95%, about 90%, about 80%, about 70%, about 60%, about 50%, about 40%, about 30%, about 20%, about 10%, about 5% or about 1%. The course of therapy may begin prior to, concurrent with, or after the onset of clinical symptoms of abnormal Ca²⁺ sparks and its complications. Thus, the subject may have abnormal Ca²⁺ sparks and its complications, or merely be susceptible to abnormal Ca²⁺ sparks and its complications. The results of the inhibition may be permanent or may continue for a period of days (such as 1, 2, 3, 4, 5, 6 or 7 days), weeks (such as 1, 2, 3 or 4 weeks) or months (such as 1, 2, 3, 4, 5, 6 or more months).

The antibodies and formulations may be administered to a subject using different schedules, depending on the particular aim or goal of the method; the age and size of the subject; and the general health of the subject, to name only a few factors to be considered. In general, the antibodies and formulations may be administered once, or twice, three times, four times, five times, six times or more, over a course of treatment, inhibition, or prevention. The timing between each dose in a dosing schedule may range between days, weeks, months, or years, an includes administered once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more weeks. The same quantity of antibody may be administered in each dose of the dosing schedule, or the amounts in each dose may vary. The identity of the particular antibody may also vary or remain the same in each dose in a dosing schedule.

In each of the methods of the present invention, an “effective amount” of an antibody or a pharmaceutical formulation comprising an antibody is administered to a subject. The effective amount will vary between subjects. However, the effective amount is one that is sufficient to achieve the aim or goal of the method, whether inhibiting, treating or preventing. As an example, an effective amount of an antibody used in the methods of the invention is typically between about 0.1 μg to about 1000 μg of antibody per kg of body weight of the subject to which the antibody is administered. An effective amount also includes between about 1 μg to about 500 μg, between about 10 μg to about 200 μg, between about 1 μg to about 800 μg, between about 10 μg to about 800 μg, between about 1 μg to about 300 μg, and between about 10 μg to about 300 μg of antibody per kg of body weight of the subject.

Appropriate doses and dosing schedules can readily be determined by techniques well known to those of ordinary skill in the art without undue experimentation. Such a determination will be based, in part, on the tolerability and efficacy of a particular dose.

Administration of the antibody or formulation may be via any of the means commonly known in the art of antibody delivery. Such routes include intravenous, intraperitoneal, intramuscular, subcutaneous and intradermal routes of administration, as well as nasal application, by inhalation, ophthalmically, orally, rectally, vaginally, or by any other mode that results in the antibody or formulation contacting mucosal tissues.

The term “subject” is intended to mean an animal, such as birds or mammals, including humans and animals of veterinary or agricultural importance, such as dogs, cats, horses, sheep, goats, and cattle.

A kit comprising the necessary components for practicing the methods of the invention, including an antibody or a pharmaceutical formulation comprising an antibody and instructions for its use, is also within the purview of the present invention.

It is understood that the examples and embodiments described herein are for illustrative purposes only and that various modifications or changes in light thereof will be suggested to persons skilled in the art and are to be included within the spirit and purview of this application and scope of the appended claims.

All documents, papers and published materials referenced herein, including books, journal articles, manuals, patent applications, published patent applications and patents, are expressly incorporated herein by reference in their entirety.

### EXAMPLES

### Inhibition of Abnormal Ca²⁺ Sparks

Materials: Animal mode Sprague-Dawley rats, isolated cardiac myocytes, NKA antibody activator, Ca²⁺ indicator fluo-4-AM, and a Zeiss LSM510 confocal microscope. Method-1. Preparation of isolated ventricular cardiac myocytes: Ventricular cardiac myocytes were isolated from adult Sprague-Dawley rat hearts. Animal weight was between 230-300 g and 2-3 months old. Following anesthesia (sodium pentobarbital, 100 mg/kg, intraperitoneal injection), the heart was removed from the animal chest and immediately perfused via the aorta using the Langendorff method and collagenase (Worthington Type II, 1 mg/ml). The perfusion was terminated when animal heart tissue became soft (25-30 minutes). Single cells were then gently shaken to loosen from the heart and suspended and stored in Hepes-buffered solution containing (mM): 137 NaCl, 5.4 KCl, 1.2 MgCl2, 1 NaH2PO4, 1 CaCl2, 20 glucose and 20 Hepes (pH 7.4) at 37° C. in a CO2 incubator until used.

Method-2. Confocal Ca²⁺ sparks detection: Isolated single cardiac myocytes were first incubated with Ca²⁺ indicator fluo-4-AM (15 AM) for 5 minutes, followed by 10 minute de-esterification of the Ca²⁺ indicator. Isolated cardiac myocytes were then placed on the stage, and confocal imaging was performed in the presence and absence of the NKA antibody activator SSA412. Line scan images were acquired using a confocal microscope Zeiss LSM510 (Carl Zeiss Inc., Germany) equipped with an argon laser (488 nm) and equipped with an argon laser (488 nm) and a 40×, 1.3 NA oil immersion objective, at axial and radial resolutions of 1.0 and 0.4 μm, respectively. The line scan (x-t) imaging was performed with space-time sampling rates of 0.77 ms per scan line and 0.045 μm per pixel.

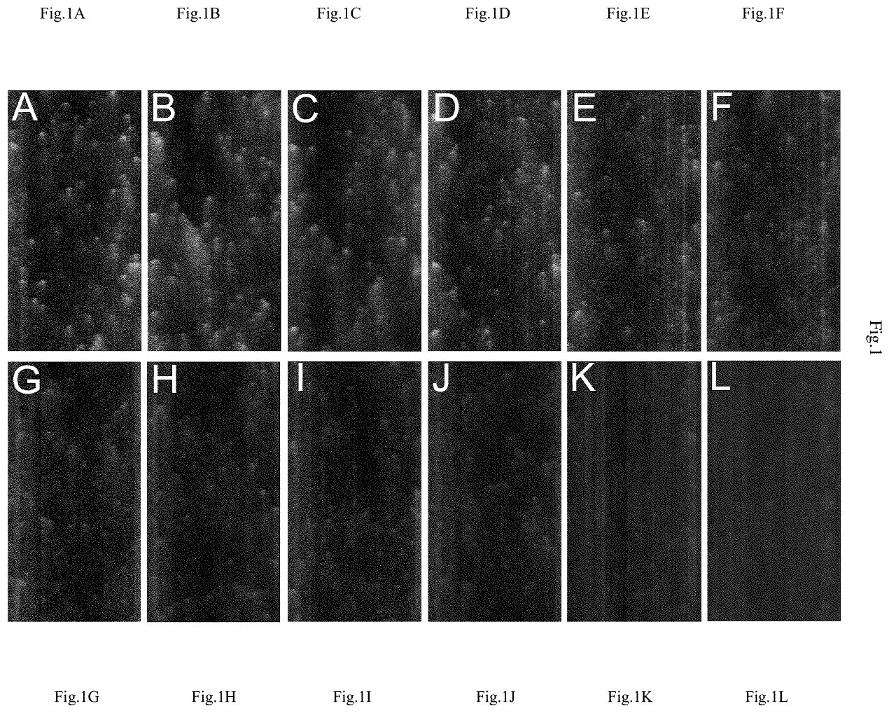

Method-3. NKA antibody activator inhibits abnormal Ca²⁺ sparks and its complications: Confocal time course images reveal that abnormal Ca²⁺ sparks gradually are inhibited and disappeared in the presence of NKA antibody activator SSA412 compared with the absence of NKA activator (FIG. 1A) as shown in FIG. 1C, FIG. 1D, FIG. 1E, FIG. 1F, FIG. 1G, FIG. 1H, FIG. 1I, FIG. 1J, FIG. 1K, & FIG. 1L. These results provide direct evidence demonstrating that NKA antibody activator SSA412 significantly inhibited massive abnormal Ca²⁺ sparks and its induced Ca²⁺ overload and dysfunction of cell Ca²⁺ homeostasis complications. FIG. 1 demonstrates that the NKA antibody activator-based immunotherapy has a therapeutic effect on inhibiting, treating, and reversing abnormal Ca²⁺ sparks, which protect cardiac myocyte cell function, maintain cellular Ca²⁺ homeostasis, reverse abnormal Ca²⁺ sparks condition, and abnormal Ca²⁺ sparks-induced complications.

What is claimed is:

1. A method for inhibiting abnormal calcium sparks in a subject comprising administering an effective amount of an antibody having binding specificity for α or β subunit of NKA to a subject in need thereof, wherein the antibody has binding specificity for one or more of the peptides represented by SEQ ID NOs: 3-7.

2. The method of claim 1, wherein the antibody is antibody SSA78 or a humanized or human version thereof, or a fragment or derivative thereof.

3. The method of claim 1, wherein the antibody is antibody SSA401 or a humanized or human version thereof, or a fragment or derivative thereof.

4. The method of claim 1, wherein the antibody is antibody SSA412 or a humanized or human version thereof, or a fragment or derivative thereof.

5. The method of claim 1, wherein the antibody is antibody JY2948 or a humanized or human version thereof, or a fragment or derivative thereof.

6. The method of claim 1, wherein the antibody is antibody JY421228 or a humanized or human version thereof, or a fragment or derivative thereof.

7. The method of claim 1, wherein the antibody is in a pharmaceutical formulation comprising the antibody and a pharmaceutically acceptable carrier.

8. The method of claim 1, wherein the subject has or is at greater risk than the general population for a disease or condition selected from the group consisting of one or more of the following diseases and conditions: abnormal calcium sparks and its induced/regulated life-threatening ventricular arrhythmia, ventricular tachycardia, ventricular fibrillation, prevalent atrial arrhythmias (atrial fibrillation and flutter), heart failure, myocardial infarction, SR calcium leak, muscular dystrophy, calcium overload, diabetes associated calcium signaling dysfunction, hypertension, dysfunction of calcium homeostasis in myocardial ischemia/reperfusion injury, abnormal calcium sparks induced-stroke, and abnormal calcium sparks in aged and dying cells.

9. A method for inhibiting abnormal calcium sparks induced complications in a subject comprising administering an effective amount of an antibody having binding specificity for the α or β subunit of NKA to a subject in need thereof. Wherein the antibody has binding specificity for one or more of the peptides represented by SEQ ID NOs: 3-7.

10. The method of claim 9, wherein the antibody is antibody SSA78 or a humanized or human version thereof, or a fragment or derivative thereof.

11. The method of claim 9, wherein the antibody is antibody SSA401 or a humanized or human version thereof, or a fragment or derivative thereof.

12. The method of claim 9, wherein the antibody is antibody SSA412 or a humanized or human version thereof, or a fragment or derivative thereof.

13. The method of claim 9, wherein the antibody is antibody JY2948 or a humanized or human version thereof, or a fragment or derivative thereof.

14. The method of claim 9, wherein the antibody is antibody JY421228 or a humanized or human version thereof, or a fragment or derivative thereof.

15. The method of claim 9, wherein the antibody is in a pharmaceutical formulation comprising the antibody and a pharmaceutically acceptable carrier.

16. The method of claim 9, wherein the subject has or is at greater risk than the general population for a disease or condition selected from the group consisting of wherein the subject has or is at greater risk than the general population for a disease or condition selected from the group consisting of abnormal calcium sparks and its induced/regulated life-threatening ventricular arrhythmia, ventricular tachycardia, ventricular fibrillation, prevalent atrial arrhythmias (atrial fibrillation and flutter), heart failure, myocardial infarction, SR calcium leak, muscular dystrophy, calcium overload, diabetes associated calcium signaling dysfunction, hypertension, dysfunction of calcium homeostasis in myocardial ischemia/reperfusion injury, abnormal calcium sparks induced-stroke, and abnormal calcium sparks in aged and dying cells.

17. A method for inhibiting and treating abnormal calcium sparks and its complications in a subject comprising administering an effective amount of a peptide antigen vaccine for generating antibodies having binding specificity for the α or β subunit of NKA to a subject in need thereof.

18. The method of claim 17, wherein the peptides represented by SEQ ID NOs: 3-7 and its derivatives.

19. The method of claim 17, wherein the peptide antigen is in a pharmaceutical formulation comprising the peptide antigen and a pharmaceutically acceptable carrier.

20. The method of claim 17, wherein the disease is selected from the group consisting of abnormal calcium sparks, life-threatening ventricular arrhythmia, ventricular tachycardia, ventricular fibrillation, prevalent atrial arrhythmias (atrial fibrillation and flutter), heart failure, myocardial infarction, SR calcium leak, muscular dystrophy, calcium overload, diabetes associated calcium signaling dysfunction, hypertension, dysfunction of calcium homeostasis in myocardial ischemia/reperfusion injury, and abnormal calcium sparks induced stroke. Abnormal Ca²⁺ sparks induced stroke, and abnormal calcium sparks in aged and dying cells.


SEQUENCE LISTING (17 sequences)

SEQ ID NO: 1 - protein, 15 aa, synthetic construct
  REGION 1..15: note = Activation site of the (Na+K)-ATPase
KRQPRNPKTDKLVNE

SEQ ID NO: 2 - protein, 13 aa, synthetic construct
  REGION 1..13: note = Activation site of the (NA+K)-ATPase
VPASLAYEQAESD

SEQ ID NO: 3 - protein, 12 aa, synthetic construct
  REGION 1..12: note = Activation site of the (Na+K)-ATPase
RSATEEEPPNDD

SEQ ID NO: 4 - protein, 15 aa, synthetic construct
  REGION 1..15: note = Activation site of the (Na+K)-ATPase
HLLGIRETWDDRWIN

SEQ ID NO: 5 - protein, 15 aa, synthetic construct
  REGION 1..15: note = Activation site of the (Na+K)-ATPase
DVEDSYGQQWTYEQR

SEQ ID NO: 6 - protein, 13 aa, synthetic construct
  REGION 1..13: note = Activation site of the (Na+K)-ATPase
KERGEFNHERGER

SEQ ID NO: 7 - protein, 13 aa, synthetic construct
  REGION 1..13: note = Activation site of the (Na+K)-ATPase
RDEDKDKVGNIEY

SEQ ID NO: 8 - protein, 122 aa, synthetic construct
  REGION 1..122: note = Mouse monoclonal antibody
QVQLQQPGAELVKPGASVKLSCKASGYTFTSYWMHWVKQRPGQGLEWIGTINPHSGSSYYSEKFKSKATLTADTSSSTAYMQLSSLTSEDSAVYYCTAVAGGYYDQGALDYWGRGTSVTVSS

SEQ ID NO: 9 - protein, 112 aa, synthetic construct
  REGION 1..112: note = Mouse monoclonal antibody
DIVMSQSPSSLAVSAGEKVTMSCKSSQSLFNSRTRKNYLAWYQQKPGQSPKLLIYWASTRESGVPDRFTGSGSGTDFTLTISSVQAEDLAVYYCKQYSYLRTFGGGTKLEIK

SEQ ID NO: 10 - protein, 122 aa, synthetic construct; identical to SEQ ID NO: 8
  REGION 1..122: note = Mouse monoclonal antibody

SEQ ID NO: 11 - protein, 112 aa, synthetic construct; identical to SEQ ID NO: 9
  REGION 1..112: note = Mouse monoclonal antibody

SEQ ID NO: 12 - protein, 122 aa, synthetic construct; identical to SEQ ID NO: 8
  REGION 1..122: note = Mouse monoclonal antibody

SEQ ID NO: 13 - protein, 112 aa, synthetic construct; identical to SEQ ID NO: 9
  REGION 1..112: note = mouse monoclonal antibody

SEQ ID NO: 14 - protein, 122 aa, synthetic construct; identical to SEQ ID NO: 8
  REGION 1..122: note = mouse monoclonal antibody

SEQ ID NO: 15 - protein, 112 aa, synthetic construct; identical to SEQ ID NO: 9
  REGION 1..112: note = mouse monoclonal antibody

SEQ ID NO: 16 - protein, 122 aa, synthetic construct; identical to SEQ ID NO: 8
  REGION 1..122: note = mouse monoclonal antibody

SEQ ID NO: 17 - protein, 112 aa, synthetic construct; identical to SEQ ID NO: 9
  REGION 1..112: note = mouse monoclonal antibody